(19) United States
(12) Patent Application Publication
(10) Pub. No.: US 2008/0113969 A1
(43) Pub. Date: May 15, 2008

(54) COMBINATION THERAPY FOR THE TREATMENT OF PAIN

(76) Inventor: James Conrad Eisenach, Winston Salem, NC (US)

Correspondence Address: Christopher A. Klein;King Pharmaceuticals, Inc., 400 Crossing Blvd., Bridgewater, NJ, 08807, US

(21) Appl. No.: 11/872,878

(22) Filed: Oct. 16, 2007

Related U.S. Application Data
(60) Provisional application No. 60/852,815, filed on Oct. 19, 2006.

(51) Int. Cl. A61K 31/485; A61K 31/381; A61K 31/498; A61K 31/5513; A61K 31/4725; A61K 31/444; A61K 31/437; A61P 25/04

(52) U.S. Cl. 514/220; 514/443; 514/282; 514/249; 514/309; 514/334; 514/292

(57) ABSTRACT

The present invention provides synergistic combinations for the treatment of conditions associated with pain including acute pain, e.g., postoperative pain, chronic pain, inflammatory pain, neuropathic pain and pain associated with migraine. In particular, the present invention relates to the use of an allosteric adenosine A₁ receptor enhancer in conjunction with opioid analgesics or 2-amino-3-hydroxy-5-methyl-4-isoxazole-propionic acid (AMPA)/kainate antagonists for alleviating pain, e.g., postoperative pain.

This application claims the benefit of U.S. Provisional Application No. 60/852,815, filed Oct. 19, 2006, incorporated herein by reference in its entirety.

### FIELD OF THE INVENTION

The present invention provides synergistic combinations for the treatment of conditions associated with pain, including acute pain, e.g., postoperative pain, chronic pain, inflammatory pain, neuropathic pain and pain associated with migraine. In particular, the present invention relates to the use of an allosteric adenosine A₁ receptor enhancer in conjunction with opioid analgesics or 2-amino-3-hydroxy-5-methyl-4-isoxazole-propionic acid (AMPA)/kainate antagonists for alleviating pain, e.g., postoperative pain.

### BACKGROUND OF THE INVENTION

In the past two decades, postoperative pain has received relatively little attention compared to acute inflammation or chronic nerve injury pain. Although, postoperative pain has been argued to be easily treated, repeated surveys continue to demonstrate a high incidence of severe pain. For severe postoperative pain, opioids remain the most commonly used agents. However, their unwanted side-effects, including postoperative nausea and vomiting, drowsiness, respiratory depression, and gastrointestinal and bladder dysfunction, limit opioid dosing for postoperative pain. Other potential postoperative analgesics such as α2 adrenergic receptor agonists and glutamate receptor antagonists are also accompanied with bothersome side effects. Therefore, it is desirable to develop new medicines which can help patients easily to recover after surgery with early mobilization and more rapid discharge.

Adenosine is an endogenous nucleoside present in all cell types of the body. It is endogenously formed and released into the extracellular space under physiological and pathophysiological conditions characterized by an increased oxygen demand/supply ratio. This means that the formation of adenosine is accelerated in conditions with increased high energy phosphate degradation. The biological actions of adenosine are mediated through specific adenosine receptors located on the cell surface of various cell types, including nerves. The hyper-reactive nerves increase adenosine release due to an increase in metabolic activity.

Adenosine A₁ receptors are widely distributed in most species and mediate diverse biological effects. The following examples are intended to show the diversity of the presence of A₁ receptors rather than a comprehensive listing of all such receptors. A₁ receptors are particularly ubiquitous within the central nervous system (CNS), with high levels being expressed in the cerebral cortex, hippocampus, cerebellum, thalamus, brain stem, and spinal cord. Immuno-histochemical analysis using polyclonal antisera generated against rat and human adenosine A₁ receptors has identified different labeling densities of individual cells and their processes in selected regions of the brain. Adenosine A₁ receptor mRNA is widely distributed in peripheral tissues such as the vas deferens, testis, white adipose tissue, stomach, spleen, pituitary, adrenal, heart, aorta, liver, eye and bladder. Only very low levels of A₁ receptors are thought to be present in lung, kidney, and small intestine.

Adenosine has been proposed as treatment for pain states derived from nociception including acute pain, tissue injury pain and nerve injury pain. Adenosine modulates the pain response by stimulating adenosine A₁ receptors present in the dorsal root of the spinal cord and higher brain centers (spraspinal mechanisms). Adenosine A₁ agonists have been shown to be effective treatment for pain in animal pain models. However, A₁ agonists also cause cardiovascular side effects and CNS side effects such as heart block, hypotension and sedation.

### SUMMARY OF THE INVENTION

The present invention relates to a class of compounds known as allosteric adenosine A₁ receptor modulators or allosteric adenosine A₁ receptor enhancers.

The activation of adenosine A₁ receptors by the allosteric adenosine A₁ receptor enhancer (2-amino-4,5,6,7-tetrahydrobenzo[b]thiophen-3-yl)(4-chlorophenyl)methanone of the formula

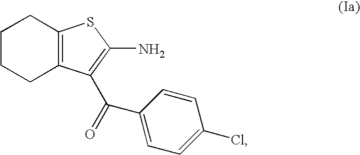

also known as T-62, has been previously demonstrated to reduce inflammatory and neuropathic pain and shown to be orally effective and avoid of the adverse side effects associated with administration of adenosine (Li et al., J. Pharmacol. Exp. Ther. 2003, 305, 950-955; Li et al., Pain 2002, 97, 117-125; Pan et al. Anesthesiology 2001, 95, 416-420; U.S. Pat. No. 6,248,774 and No. 6,489,356). Although, the effect of T-62 in postincisional hypersensitivity in rats is modest, it has now been surprisingly discovered that T-62 interacts synergistically with opioids and AMPA/kainate antagonists in alleviating pain, in particular postoperative pain. The entire contents of U.S. Pat. No. 6,248,774 and No. 6,489,356 are incorporated herein by reference.

Accordingly, the present invention relates to a combination, such as a combined preparation or a pharmaceutical composition, respectively, for achieving a synergistic therapeutic effect comprising alleviating pain, including acute pain, e.g., postoperative pain, chronic pain, inflammatory pain, neuropathic pain and pain associated with migraine, in a warm-blooded animal, including man, in need thereof, which combination comprises synergistic amounts of an allosteric adenosine A₁ receptor enhancer, or a pharmaceutically acceptable salt thereof, and another therapeutic agent selected from the group consisting of:

(1) an opioid, or a pharmaceutically acceptable salt thereof; and

(2) an AMPA/kainate antagonist, or a pharmaceutically acceptable salt thereof.

In another aspect, the present invention relates to a method for achieving a synergistic therapeutic effect comprising alleviating pain, including acute pain, e.g., postoperative pain, chronic pain, inflammatory pain, neuropathic pain and pain associated with migraine, in a warm-blooded animal, including man, in need thereof, which method comprises administering to said warm-blooded animal synergistic amounts of an allosteric adenosine A₁ receptor enhancer, or a pharmaceutically acceptable salt thereof, and another therapeutic agent selected from the group consisting of:

(1) an opioid, or a pharmaceutically acceptable salt thereof; and

(2) an AMPA/kainate antagonist, or a pharmaceutically acceptable salt thereof.

Other objects, features, advantages and aspects of the present invention will become apparent to those skilled in the art from the following description and appended claims. It should be understood, however, that the following description, appended claims, and specific examples, while indicating preferred embodiments of the invention, are given by way of illustration only. Various changes and modifications within the spirit and scope of the disclosed invention will become readily apparent to those skilled in the art from reading the following. Abbreviations are those generally known in the art.

### BRIEF DESCRIPTION OF DRAWINGS

FIG. 1 shows a graphical presentation of the anti-hypersensitivity effect of the allosteric adenosine A₁ receptor enhancer T-62 in postoperative pain model in adult male rats (n=7-14 for each dose);

FIG. 2 shows a graphical presentation of the anti-hypersensitivity effect of the opioid morphine in postoperative pain model in adult male rats (n=7-8 for each dose);

FIG. 3 shows a graphical presentation of the anti-hypersensitivity effect of a combination of T-62 and morphine in postoperative pain model in adult male rats (n=6-7 for each dose);

FIG. 4 shows a graphical presentation of the anti-hypersensitivity effect of the AMPA/kainate antagonists 2,3-dihydroxy-6-nitro-7-sulfamoylbenzo(f)quinoxaline (NBQX) in postoperative pain model in adult male rats (n=6-7 for each dose);

FIG. 5 shows a graphical presentation of the anti-hypersensitivity effect of a combination of T-62 and NBQX in postoperative pain model in adult male rats (n=6-7 for each dose);

FIG. 6 shows an isobologram of the combination of T-62 with morphine which demonstrates a synergistic interaction between T-62 and morphine;

FIG. 7 shows an isobologram of the combination of T-62 with NBQX which demonstrates a synergistic interaction between T-62 and NBQX; and

FIG. 8 shows a graphical presentation of the anti-hypersensitivity effects of three allosteric adenosine A₁ receptor enhancers in neuropathic pain model in male Spraque-Dawley rats, i.e., T-62 and compounds of formulae (Ib) and (Ic).

### DETAILED DESCRIPTION OF THE INVENTION

As described above, the present invention provides synergistic combinations for the treatment of conditions associated with pain, including acute pain, e.g., postoperative pain, chronic pain, inflammatory pain, neuropathic pain and pain associated with migraine. In particular, the present invention relates to the use of an allosteric adenosine A₁ receptor enhancer in conjunction with opioid analgesics or AMPA/kainate antagonists for alleviating pain, e.g., postoperative pain.

Listed below are some of the definitions of various terms used herein to describe certain aspects of the present invention. However, the definitions used herein are those generally known in the art and apply to the terms as they are used throughout the specification unless they are otherwise limited in specific instances.

The term “allosteric adenosine A₁ receptor modulator” or “allosteric adenosine A₁ receptor enhancer” as used herein refers to a class of compounds that appear to enhance adenosine A₁ receptor function by stabilizing the high affinity state of the receptor-G-protein complex. This property may be measured as an increase in radioligand binding to the adenosine A₁ receptor. An enhancer that increases agonist binding can do so by either accelerating the association of an agonist to the receptor, or by retarding the dissociation of the “receptor-ligand” complex and, therefore, must bind to a site different from the agonist recognition site. This putative site is termed as the allosteric site, and presumably, compounds that bind to this site and enhance the agonist effect are termed as “allosteric enhancers”.

The term “AMPA/kainate antagonist” as used herein refers to both competitive and non-competitive AMPA receptor antagonists which may additionally exhibit antagonist activity towards the other glutamate receptors, e.g., the kainate receptors.

The term “therapeutically effective amount” refers to an amount of a drug or a therapeutic agent that will elicit the desired biological or medical response of a tissue, system or an animal (including man) that is being sought by a researcher or clinician, e.g., provides significant analgesic activity.

The term “treatment” shall be understood as the management and care of a patient for the purpose of combating the disease, condition or disorder.

The term “pain-alleviating” shall be understood herein to include the expressions “pain-suppressing”, “pain-reducing” and “pain-inhibiting” as the present invention is applicable to the alleviation of existing pain as well as the suppression or inhibition of pain which would otherwise ensue from an imminent pain-causing event.

The term “synergistic” or “potentiate” as used herein, means that the effect achieved with the methods, combinations and pharmaceutical compositions of the present invention is greater than the sum of the effects that result from individual methods and compositions comprising the active ingredients of the present invention separately.

The term “warm-blooded animal, mammal or patient” are used interchangeably herein and include, but are not limited to, humans, dogs, cats, horses, pigs, cows, monkeys, rabbits, mice and laboratory animals. The preferred mammals are humans.

The term “pharmaceutically acceptable salt” refers to a non-toxic salt commonly used in the pharmaceutical industry which may be prepared according to methods well-known in the art.

The term “combination” of an allosteric adenosine A₁ receptor enhancer, in particular T-62, and another therapeutic agent referred to herein above, or in each case, a pharmaceutically acceptable salt thereof, means that the components can be administered together as a pharmaceutical composition or as part of the same, unitary dosage form. A combination also includes administering an allosteric adenosine A₁ receptor enhancer, in particular T-62, or a pharmaceutically acceptable salt thereof, and another therapeutic agent referred to herein above, or a pharmaceutically acceptable salt thereof, each separately but as part of the same therapeutic regimen. The components, if administered separately, need not necessarily be administered at essentially the same time, although they can if so desired. Thus, a combination also refers, for example, administering an allosteric adenosine A₁ receptor enhancer, in particular T-62, or a pharmaceutically acceptable salt thereof, and another therapeutic agent, or a pharmaceutically acceptable salt thereof, as separate dosages or dosage forms, but at the same time. A combination also includes separate administration at different times, in any order and by any route of administration.

The allosteric adenosine A₁ receptor enhancers to which the present invention applies are any of those enhancing the function of adenosine A₁ receptors in vivo and, therefore, having pharmaceutical utility, e.g., as therapeutic agents for reducing pain, including acute pain, e.g., postoperative pain, chronic pain, inflammatory pain, neuropathic pain and pain associated with migraine, in particular postoperative pain.

Suitable allosteric adenosine A₁ receptor enhancers include, but are not limited to, 2-amino-3-acylthiophene derivatives, e.g., those disclosed in U.S. Pat. No. 6,323,214 and No. 6,727,258, the entire contents of which are incorporated herein by reference.

Preferably, the allosteric adenosine A₁ receptor enhancer of the present invention is selected from the group consisting of the compound T-62 of the formula

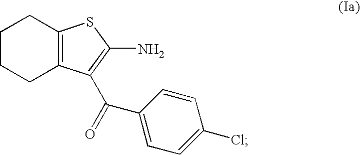

the compound of the formula

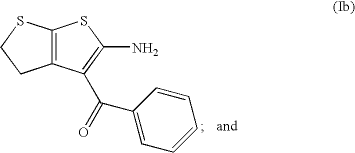

the compound of the formula

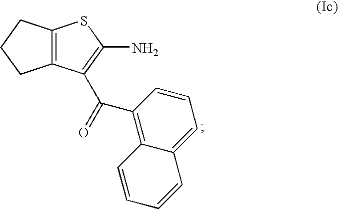

or in each case, a pharmaceutically acceptable salt thereof.

More preferably, the allosteric adenosine A₁ receptor enhancer of the present invention is the compound T-62 of formula (Ia).

The allosteric adenosine A₁ receptor enhancers of the present invention may be prepared using methods well known in the art, e.g., the compounds of formulae (Ia), (Ib) and (Ic) may be prepared using methods disclosed in U.S. Pat. No. 6,323,214 and No. 6,727,258, or as described by Corral et al. in Afinidad 1978, 35(354), 129-33.

A variety of opioids have been described in the art, e.g., those generally used as pain relievers, narcotics and/or anesthetics and include, but are not limited to, alfentanil, allylprodine, alphaprodine, anileridine, apomorphine, apocodeine, benzylmorphine, bezitramide, buprenorphine, butorphanol, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, nalbuphine, narceine, nicomorphine, norlevorphanol, normethadone, nalorphine, normorphine, norpipanone, ohmefentanyl, opium, oxycodone, oxymorphone, papavereturn, pentazocine, phenadoxone, phenomorphan, phenazocine, phenoperidine, pholcodine, piminodine, piritramide, propheptazine, promedol, profadol, properidine, propiram, propoxyphene, remifentanyl, sufentanyl, tramadol, tilidine, naltrexone, naloxone, nalmefene, methylnaltrexone, naloxone methiodide, nalorphine, naloxonazine, nalide, nalmexone, nalbuphine, nalorphine dinicotinate, naltrindole (NTI), naltrindole isothiocyanate, (NTII), naltriben (NTB), nor-binaltorphimine (nor-BNI), β-funaltrexamine (β-FNA), cyprodime, etorphine, diprenorphine, naloxone benzoylhydrazone, bremazocine, ethylketocyclazocine, spiradoline, Met-enkephalin, Leu-enkephalin, β-endorphin, dynorphin A, dynorphin B or α-neoendorphin, or a pharmaceutically acceptable salt thereof.

Preferred opioids for the combination and use according to the present invention include hydrocodone, hydromorphone, morphine, oxycodone and oxymorphone, in particular morphine, or in each case, a pharmaceutically acceptable salt thereof.

Suitable AMPA/kainate antagonists include, but are not limited to, antagonists such as NBQX (2,3-dihydroxy-6-nitro-7-sulfamoylbenzo[f]quinoxaline), CNQX (6-cyano-7-nitro-quinoxaline-2,3-dione, DNQX (6,7-dinitroquinoxaline-2,3-dione), PNQX (1,4,7,8,9,10-hexahydro-9-methyl-6-nitropyrido[3,4-f]quinoxaline-2,3-dione), and compounds disclosed in U.S. Pat. No. 6,117,873, e.g., becampanel of the formula

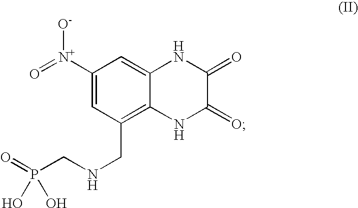

those disclosed in U.S. Pat. No. 5,536,832, e.g., talampanel of the formula

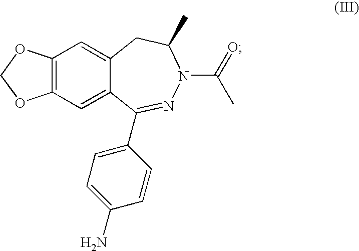

those disclosed in U.S. Pat. No. 5,670,516, e.g., tezampanel of the formula

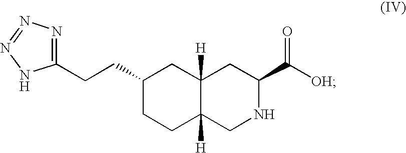

those disclosed in U.S. Pat. No. 6,949,571, e.g., perampanel of the formula

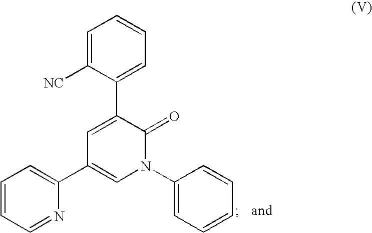

those disclosed in U.S. Pat. No. 6,693,111, e.g., NS-1209 of the formula

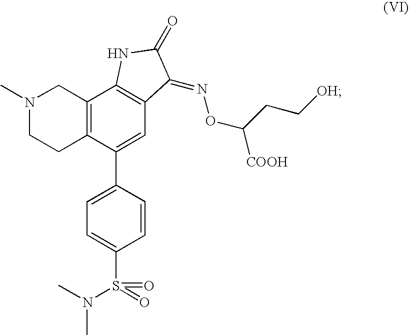

or in each case, a pharmaceutically acceptable salt thereof. The entire contents of U.S. Pat. No. 6,117,873; No. 5,536,832; No. 5,670,516; No. 6,949,571; and No. 6,693,111 are incorporated herein by reference.

As referred to herein above, the allosteric adenosine A₁ receptor enhancers of the present invention, and the combination partners thereof, may be present as their pharmaceutically acceptable salts. If these compounds have, e.g., at least one basic center such as an amino group, they can form acid addition salts thereof. Similarly, the compounds having at least one acid group (for example —COOH) can form salts with bases.

The corresponding active ingredients, or a pharmaceutically acceptable salts, may also be used in the form of a solvate, such as a hydrate, or including other solvents used, e.g., those employed in their crystallization.

Preferably, a combination according to the present invention comprises an allosteric adenosine A₁ receptor enhancer, e.g., T-62, or a pharmaceutically acceptable salt thereof, and an opioid, e.g., hydrocodone, hydromorphone, morphine, oxycodone or oxymorphone, in particular morphine, or in each case, a pharmaceutically acceptable salt thereof.

Preferred is also a combination according to the present invention which comprises an allosteric adenosine A₁ receptor enhancer, e.g., T-62, or a pharmaceutically acceptable salt thereof, and an AMPA/kainate antagonist, e.g., NBQX, becampanel, talampanel, tezampanel, perampanel or NS-1209, or in each case, a pharmaceutically acceptable salt thereof.

The structure of the active agents identified by generic or tradenames may be taken from the actual edition of the standard compendium “The Merck Index”, or the Physician's Desk Reference, or from databases, e.g., Patents International (e.g., IMS World Publications) or Current Drugs. Accordingly, any person skilled in the art is fully enabled to identify the active agents of the present invention. Likewise, a person skilled in the art is fully enabled to manufacture and test the pharmaceutical indications and properties in standard test models known in the art, both in vitro and in vivo.

For example, drug efficacy may be assessed using pain models such as carrageenan model (Guilbaud and Kayser, Pain 1987, 28, 99-107) for acute inflammatory pain; FCA model (Freund's Complete Adjuvant; Hay et al., Neuroscience 1997, 78(3), 843-850) for chronic inflammatory pain; CCI model (Chronic Constriction Injury; Bennett and Xie, Pain 1988, 33, 87-107) and the Chung model (Kim and Chung, Pain 1992, 50, 355-363) for neuropathic pain; or postincisional hypersensitivity model (Obata et al., Anesthesiology 2004, 100, 1258-1262) for postoperative pain.

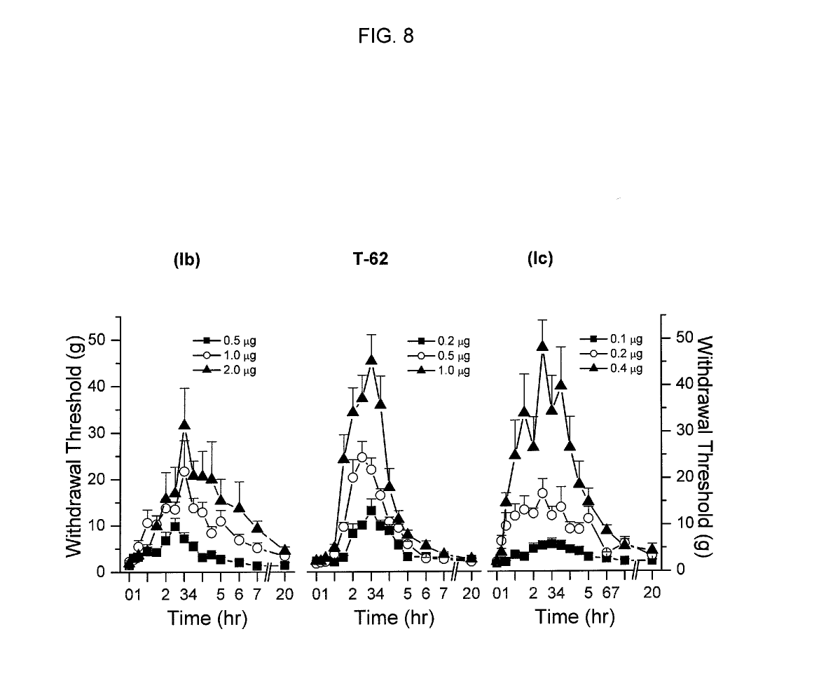

For example, as demonstrated in FIG. 8, the allosteric adenosine A₁ receptor enhancers T-62, and compounds of formulae (Ib) and (Ic), all exert dose-dependent anti-allodynic effects in the Chung model of chronic neuropathic pain, the compound of formula (Ic) being the most potent enhancer of the three agents.

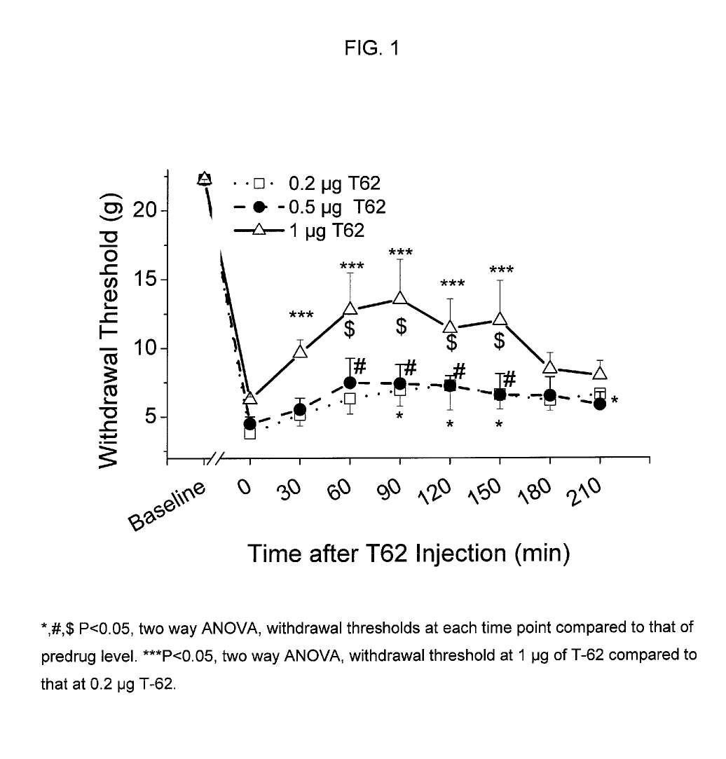

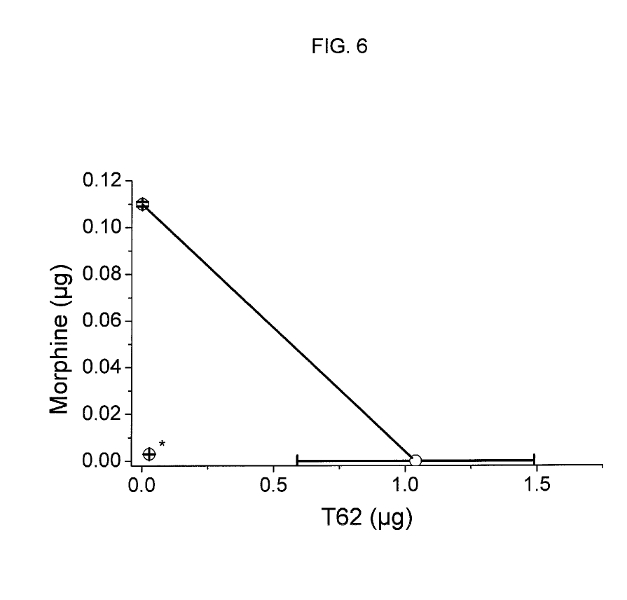

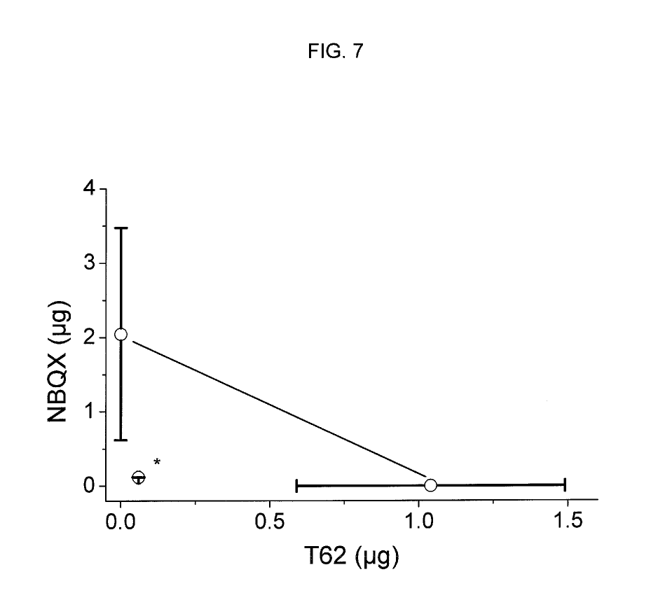

As illustrated in FIG. 1, the compound T-62 (i.t.) produces a modest effect in the postincisional hypersensitivity model of postoperative pain. However, when T-62 is combined with morphine or NBQX, FIG. 6 and FIG. 7, respectively, the observed ED₄₀ values for said combinations are 5% and 11% of the theoretical additive dose, respectively, demonstrating in each case a synergistic interaction between the drugs.

Accordingly, the present invention provides a combination, such as a combined preparation or a pharmaceutical composition, respectively, for achieving a synergistic therapeutic effect comprising alleviating pain, including acute pain, e.g., postoperative pain, chronic pain, inflammatory pain, neuropathic pain and pain associated with migraine, in a warm-blooded animal, including man, in need thereof, which combination comprises synergistic amounts of an allosteric adenosine A₁ receptor enhancer, e.g., T-62, or a pharmaceutically acceptable salt thereof, and another therapeutic agent selected from the group consisting of:

(1) an opioid, or a pharmaceutically acceptable salt thereof; and

(2) an AMPA/kainate antagonist, or a pharmaceutically acceptable salt thereof.

Likewise, the present invention provides a method for achieving a synergistic therapeutic effect comprising alleviating pain, including acute pain, e.g., postoperative pain, chronic pain, inflammatory pain, neuropathic pain and pain associated with migraine, in a warm-blooded animal, including man, in need thereof, which method comprises administering to said warm-blooded animal synergistic amounts of an allosteric adenosine A₁ receptor enhancer, e.g., T-62, or a pharmaceutically acceptable salt thereof, and another therapeutic agent selected from the group consisting of:

(1) an opioid, or a pharmaceutically acceptable salt thereof; and

(2) an AMPA/kainate antagonist, or a pharmaceutically acceptable salt thereof.

Furthermore, the present invention relates to the use of an allosteric adenosine A₁ receptor enhancer, e.g., T-62, or a pharmaceutically acceptable salt thereof, in combination with (1) an opioid; or (2) an AMPA/kainate antagonist; or in each case, a pharmaceutically acceptable salt thereof; for achieving a synergistic therapeutic effect comprising alleviating pain, including acute pain, e.g., postoperative pain, chronic pain, inflammatory pain, neuropathic pain and pain associated with migraine, in particular postoperative pain.

Likewise, the present invention relates to the use of an allosteric adenosine A₁ receptor enhancer, e.g., T-62, or a pharmaceutically acceptable salt thereof, in combination with (1) an opioid; or (2) an AMPA/kainate antagonist; or in each case, a pharmaceutically acceptable salt thereof; for the manufacture of a medicament for achieving a synergistic therapeutic effect comprising alleviating pain, including acute pain, e.g., postoperative pain, chronic pain, inflammatory pain, neuropathic pain and pain associated with migraine, in particular postoperative pain.

In one aspect, the present invention provides pharmaceutical compositions comprising an allosteric adenosine A₁ receptor enhancer, preferably T-62, or a pharmaceutically acceptable salt thereof, in an amount effective to potentiate the analgesic effect of a second therapeutic agent selected from the group consisting of:

(1) an opioid, preferably hydrocodone, hydromorphone, morphine, oxycodone or oxymorphone, in particular morphine, or in each case, a pharmaceutically acceptable salt thereof; and

(2) an AMPA/kainate antagonist, preferably NBQX, becampanel, talampanel, tezampanel, perampanel or NS-1209, or in each case, a pharmaceutically acceptable salt thereof;

and a pharmaceutically acceptable carrier; for alleviation of pain, including acute pain, e.g., postoperative pain, chronic pain, inflammatory pain, neuropathic pain and pain associated with migraine.

In another aspect, the present invention provides pharmaceutical compositions comprising: (1) an opioid, preferably hydrocodone, hydromorphone, morphine, oxycodone or oxymorphone, in particular morphine, or a pharmaceutically acceptable salt thereof; or (2) an AMPA/kainate antagonist, e.g., NBQX, becampanel, talampanel, tezampanel, perampanel or NS-1209, or a pharmaceutically acceptable salt thereof; in amount such that, following administration of a combination of the present invention, a synergistic therapeutic effect is achieved; and a pharmaceutically acceptable carrier.

In a further aspect, the present invention provides a pharmaceutical composition for achieving therapeutic effect comprising alleviating pain, including acute pain, e.g., postoperative pain, chronic pain, inflammatory pain, neuropathic pain and pain associated with migraine, in a warm-blooded animal, including man, in need thereof, which composition comprises synergistic amounts of an allosteric adenosine A₁ receptor enhancer, preferably T-62, or a pharmaceutically acceptable salt thereof, and another therapeutic agent selected from the group consisting of:

(1) an opioid, preferably hydrocodone, hydromorphone, morphine, oxycodone or oxymorphone, in particular morphine, or in each case, a pharmaceutically acceptable salt thereof; and

(2) an AMPA/kainate antagonist, preferably NBQX, becampanel, talampanel, tezampanel, perampanel or NS-1209, or in each case, a pharmaceutically acceptable salt thereof;

and a pharmaceutically acceptable carrier.

As disclosed herein above, an allosteric adenosine A₁ receptor enhancer, in particular, T-62, or a pharmaceutically acceptable salt thereof, in combination with (1) an opioid, e.g., hydrocodone, hydromorphone, morphine, oxycodone or oxymorphone, preferably morphine, or in each case a pharmaceutically acceptable salt thereof; or (2) an AMPA/kainate antagonist, e.g., NBQX, becampanel, talampanel, tezampanel, perampanel or NS-1209, or in each case, a pharmaceutically acceptable salt thereof; may be co-administered as a pharmaceutical composition. The components may be administered together in any conventional dosage form, usually also together with a pharmaceutically acceptable carrier or diluent.

In carrying out the method of the present invention, the allosteric adenosine A₁ receptor enhancers of the present invention, or the combination partners thereof, may be formulated into pharmaceutical compositions suitable for administration via a variety of routes, such as oral or rectal, transdermal, intrathecal and parenteral administration to mammals, including man. For oral administration the pharmaceutical composition comprising an allosteric adenosine A₁ receptor enhancer, in particular, T-62, or a pharmaceutically acceptable salt thereof, or a combination partner thereof, can take the form of solutions, suspensions, tablets, pills, capsules, powders, microemulsions, unit dose packets and the like. Preferred are tablets and gelatin capsules comprising the active ingredient together with: a) diluents, e.g., lactose, dextrose, sucrose, mannitol, sorbitol, cellulose, glycine and/or vegetable oil; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-90%, preferably about 1-80%, of the active ingredient(s), conveniently from 30-95% for tablets and capsules, and 3-50% for liquid preparations.

The dosage of the active ingredients can depend on a variety of factors, such as mode of administration, homeothermic species, age and/or individual condition.

Preferred dosages for the active ingredients of the pharmaceutical combinations according to the present invention are therapeutically effective dosages, especially those which are commercially available. In general, however, doses employed for adult human treatment will typically be in the range of 0.02-5000 mg/day, preferably 1-1500 mg/day, e.g., for a patient of approximately 75 kg in weight. The desired dose may conveniently be presented in a single dose or as divided doses administered at appropriate intervals, for example as two, three, four or more sub-doses per day. The dosage or dosages which will result in optimal synergistic effects is achieved by coordinating the pharmacokinetic properties, such as volume of distribution and Tₘₐₓ, of the therapeutic agents of this invention so that the therapeutic windows of each agent overlap to the maximum extent possible. Such dosages are readily determined by one skilled in the art.

For example, the doses of T-62 to be administered to warm-blooded animals, including man, of approximately 75 kg body weight, especially the doses effective for enhancing the adenosine A₁ receptor function, e.g., to alleviate pain, are from about 1 mg to about 1 g, preferably from about 10 mg to about 500 mg. Two per day are an option, one for daytime use and one for nighttime use. Since there is the potential of an allosteric adenosine A₁ receptor enhancer to cause sedation at a high dose, the higher doses are recommended for nighttime use. For example, a dose of from about 100 to about 500 mg dose of T-62 is recommended for daytime use while a dose from about 600 to about 1000 mg is recommended as a nighttime dose.

Suitable doses and dosage forms of opioids that may be employed in the combinations of the present invention include, but are not limited to, those that are commercially available, and those described in U.S. Patent Application Publication No. 2004/0161382.

Suitable doses and dosage forms of AMPA/kainate antagonists are known in the art and include, e.g., those disclosed in published preclinical and/or clinical study reports, e.g., tezampanel may be administered to a human patient in a total daily dosage of about 40 to about 100 mg (single dose, sc), and talampanel may be administered to a human patient in a total daily dosage of about 75 to about 300 mg (tid, po).

As the present invention relates to methods for alleviating pain with a combination of compounds which may be administered separately, the invention also relates to combining separate pharmaceutical compositions in a kit form. The kit may comprise, e.g., two separate pharmaceutical compositions: (1) a composition comprising an allosteric adenosine A₁ receptor enhancer, in particular T-62, or a pharmaceutically acceptable salt thereof, and a pharmaceutically acceptable carrier or diluent; and (2) a composition comprising another therapeutic agent selected from the group consisting of an opioid and an AMPA/kainate antagonist, or in each case, a pharmaceutically acceptable salt thereof, and a pharmaceutically acceptable carrier or diluent. The amounts of (1) and (2) are such that a synergistic therapeutic effect is achieved. The kit comprises a container for containing the separate compositions such as a divided bottle or a divided foil packet, wherein each compartment contains a plurality of dosage forms (e.g., tablets) comprising, e.g., (1) or (2). Alternatively, rather than separating the active ingredient-containing dosage forms, the kit may contain separate compartments each of which contains a whole dosage which in turn comprises separate dosage forms. An example of this type of kit is a blister pack wherein each individual blister contains two (or more) tablets, one (or more) tablet(s) comprising a pharmaceutical composition (1), and the second (or more) tablet(s) comprising a pharmaceutical composition (2). Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician. In the case of the instant invention a kit therefore comprises:

(1) a composition comprising an allosteric adenosine A₁ receptor enhancer, in particular T-62, or a pharmaceutically acceptable salt thereof, in an amount effective to potentiate the analgesic effect of another therapeutic agent in the kit, and a pharmaceutically acceptable carrier or diluent, in a first dosage form;
(2) a composition comprising another therapeutic agent selected from the group consisting of an opioid and an AMPA/kainate antagonist, or in each case, a pharmaceutically acceptable salt thereof, in an amount such that, following administration, a synergistic therapeutic effect is achieved, and a pharmaceutically acceptable carrier or diluent, in a second dosage form; and
(3) a container for containing said first and second dosage forms.

An allosteric adenosine A₁ receptor enhancer, e.g., T-62, or a pharmaceutical salt thereof, or the combination partners thereof, can be administered by various routes of administration. Each agent can be tested over a wide-range of dosages to determine the optimal drug level for each therapeutic agent alone, or in the specific combination thereof, to elicit the maximal response. For these studies, it is preferred to use treatment groups consisting of at least 6 animals per group. Each study is best performed in away wherein the effects of the combination treatment group are determined at the same time as the individual components are evaluated. Although drug effects may be observed with acute administration, it may be preferable to observe certain responses in a chronic setting. A long-term study, when appropriate, is of sufficient duration to allow for the full development of compensatory responses to occur and, therefore, the observed effect will most likely depict the actual responses of the test system representing sustained or persistent effect.

It has been found that a combination of an allosteric adenosine A₁ receptor enhancer, e.g., T-62, or a pharmaceutically acceptable salt thereof, with (1) an opioid; or (2) an AMPA/kainate antagonist; or in each case, a pharmaceutically acceptable salt thereof; achieves greater therapeutic effect in the treatment of conditions associated with pain and in alleviating the symptoms associated therewith than the administration of an allosteric adenosine A₁ receptor enhancer, an opioid or an AMPA/kainate antagonist alone. Greater efficacy can also be documented as a prolonged duration of action. The duration of action can be monitored as either the time to return to baseline prior to the next dose or as the area under the curve (AUC).

Further benefits are that lower doses of the individual drugs to be combined according to the present invention can be used to reduce the dosage, e.g., that the dosages need not only often be smaller but are also applied less frequently. or can be used to diminish the incidence of side effects. The combined administration of an allosteric adenosine A₁ receptor enhancer, e.g., T-62, or a pharmaceutically acceptable salt thereof, with (1) an opioid; or (2) an AMPA/kainate antagonist; or in each case, a pharmaceutically acceptable salt thereof; may also be used to diminish the incidence of side effects.

In particular, all the more surprising is the experimental finding that the combinations of the present invention results in a synergistic therapeutic effect for the treatment of pain, including acute pain, e.g., postoperative pain, chronic pain, inflammatory pain, neuropathic pain and pain associated with migraine.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein below are to be construed as merely illustrative of certain aspects of the present invention and are not a limitation of the scope of the present invention in any way.

### EXAMPLE 1

### Postincisional Hypersensitivity Model

Animal preparation: Following Animal Care and Use Committee approval, an intrathecal (i.t.) catheter with tip in the lumbar space is inserted as previously described in adult male rats weighing 250 g (Yaksh and Rudy, Physiol Behav. 1976, 7, 1032-1036). One week following the insertion of the i.t. catheter, paw incision surgery is performed as previously described (Brennan et al., Pain 1996, 64, 493-501). Briefly, animals are anesthetized with halothane, and a 1-cm long incision is made in the plantar aspect of the left hind paw starting 0.5 cm from the edge of the heel toward the toe after sterile preparation with 70% ethanol. The plantaris muscle is elevated and incised longitudinally. The wound is closed with 2 mattress sutures of 5.0 silk. Subsequently, withdrawal threshold to von Frey filaments is determined one day after surgery (Chaplan et al., J. Neurosci. Methods, 1994, 53, 55-63). For all testing but pilot dose ranging studies, investigators are blinded to study drug administration. A cut off value of 22 g is used.

Experimental Design: Twenty-four hours after paw incision surgery, single drugs and their combinations are studied: T-62, morphine and NBQX.

Single drug injection: For single drug administration, a volume containing 5 μL drug is used, followed by 10 μL of saline. The anti-hypersensitivity effects for single drugs at three doses with logarithmic spacing are studied over a 210 min observation period with testing at 30 min interval. Dose response curves are first constructed, and ED₄₀ values for single drugs are then determined at the time of peak effect.

Drug interaction: For the combination of T-62 with morphine and NBQX, two injections are performed since the time of peak effect for morphine and NBQX is 30 min earlier than that of T-62 (T-62 is injected 30 min before drug injections). Paw withdrawal thresholds are determined before surgery, before drug injection, and 30, 60, 90, 120, 150, 180, and 210 min after the second drug injection. In addition, a fixed fractional dose ratio of 1:1 (w:w) is used for the combination of T-62 with morphine and NBQX. The dose response curves are then constructed for the combined drugs, and the ED₄₀ values of the combined drugs are calculated at the time point of peak effect. Subsequently, isobolograms are constructed based on the ED₄₀ values from single and combined drug administration.

Materials: T-62 may be obtained from King Pharmaceuticals (Cart, N.C.) T-62 is dissolved in saline with 20% DMSO. Morphine may be purchased from Astra Pharmaceutical Products, Inc. (Westborough, Mass.). NBQX and the remaining chemicals may be purchased from Sigma Chemical Co. (St. Louis, Mo.). Saline is used as vehicle for morphine and NBQX.

Data analysis: Data are presented as mean ±S.E. Withdrawal threshold data are analyzed by two-way analysis of variance followed by Fisher's LSD test. P<0.05 is considered significant. Isobolograms are statistically analyzed by student T test.

### Results:

A. Anti-hypersensitivity effect of intrathecal T-62 alone:

T-62 (i.t.) reduced mechanical hypersensitivity in a time dependent manner over the 3 hour period of observation. Injection of 20% DMSO in saline had no effect on withdrawal thresholds. The time of peak effect ranged from 60 to 90 min after injection (FIG. 1). Maximum possible effects (MPEs) at 60 min are used for the calculation of ED₄₀.

B. Anti-hypersensitivity effect of intrathecal T-62 and morphine:

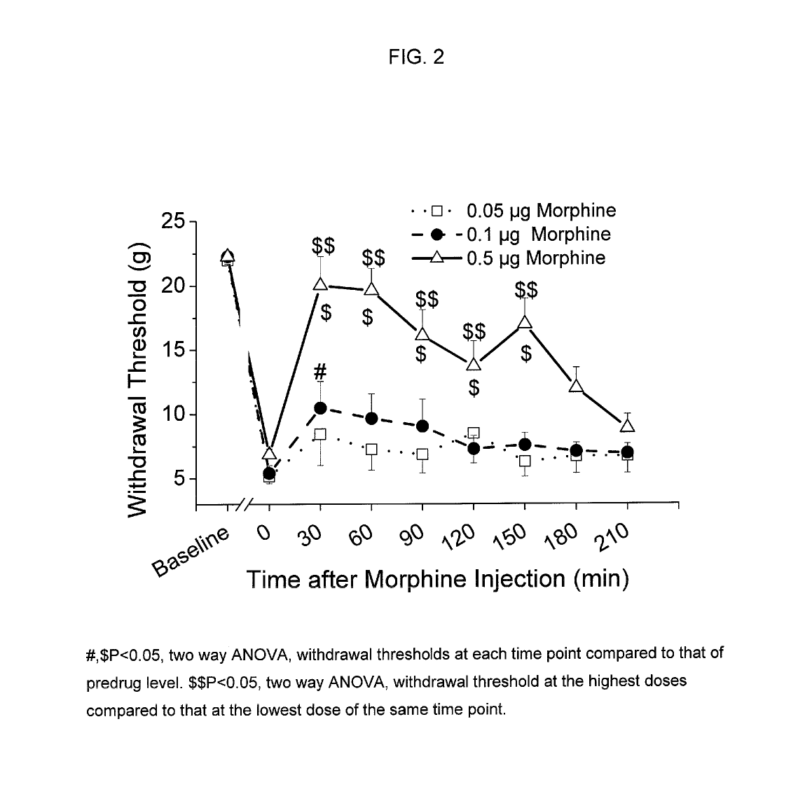

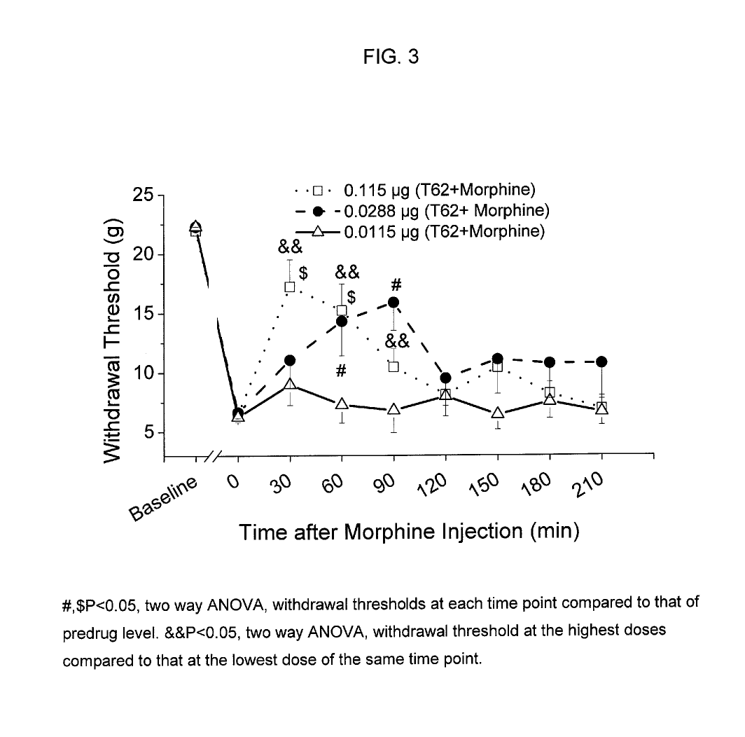

Morphine (i.t) alone reduces mechanical hypersensitivity in a time and dose dependent manner. The peak effect of i.t. morphine is reached 30 min after injection (FIG. 2). T-62 plus morphine also reduces mechanical hypersensitivity in a time and dose dependent manner (FIG. 3). Isobolographic analysis indicates a synergistic interaction with the observed ED₄₀ of combined drugs being 5% of the theoretical additive dose (FIG. 6).

C. Anti-hypersensitivity effect of intrathecal T-62 and NBQX:

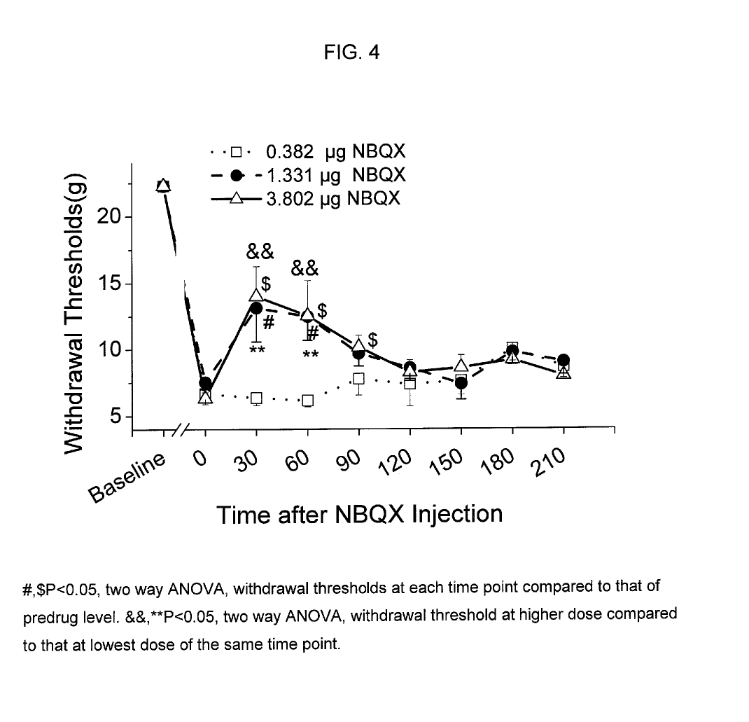

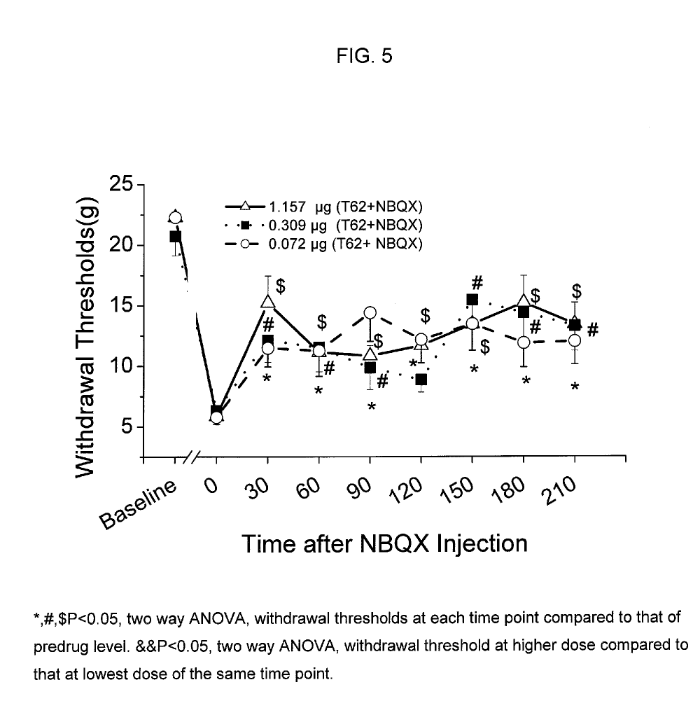

NBQX (i.t.) alone reduces the mechanical hypersensitivity in a time and dose dependent manner. The peak effect of i.t. NBQX is reached 30 min after injection (FIG. 4). T-62 plus NBQX reduces mechanical hypersensitivity in a time and dose dependent manner as well (FIG. 5). Isobolographic analysis demonstrates a synergistic interaction with the observed ED₄₀ of combined drug being 11% of the theoretical additive dose (FIG. 7).

### EXAMPLE 2

### Neuropathic Pain Model

Spinal Nerve Ligation: After Animal Care and Use Committee approval, male Sprague-Dawley rats (Harlan, Indianapolis, Ind., USA) weighing 180-200 g undergo anesthesia as previously described by Kim and Chung (Pain 1992, 50, 355-363). Briefly, under general anesthesia with inhalational halothane, the left L5 and L6 spinal nerves are identified through a small laminotomy and tightly ligated. Approximately one week later, an intrathecal catheter is placed under general anesthesia by insertion under direct vision of a polyethylene catheter through a small slit in the dura at the cisterna magnum, and advanced 8.5 cm such that the catheter tip resides in the lower lumbar intrathecal space. Animals are studied approximately one week later. Ligation of the lumbar spinal nerves results in a primarily unilateral increase in the sensitivity to light touch on the operated side. Sensitivity is assessed via application of calibrated von Frey filaments. The withdrawal threshold is determined using an up down method as previously described by Chaplan et al. (J. Neurosci. Methods 1994, 53, 55-63).

Experimental Design: Animals receive, at least 4 weeks after spinal nerve ligation surgery, and at least 1 week after intrathecal catheter insertion, single injections of vehicle (β-cyclodextrin) or the drug substance, using a randomized and blinded protocol. Withdrawal thresholds are determined at intervals for up to 20 hr after injection. The results are shown in FIG. 8. As noted in FIG. 8, all agents exhibited dose-dependent increases in withdrawal threshold, with a slow onset, and long duration of action.

### EXAMPLE 3

### T-62 Formulation A

T-62 may be obtained from King Pharmaceuticals (Cary, N.C.) in dry powder form. T-62 is screened through a #40 screen and then added to a mixture of propylene glycol monocaprylate (Capryol 90®), caprylocaproyl macrogol-8 glycerides (Labrasol®), super refined soybean oil (USP) and polysorbate 80 (Crillet 4 HP®) at 50° C. (±5° C.) while mixing with a propeller mixer to dissolve T-62. The mixture/solution is sparged with nitrogen throughout the process. The resulting solution has a density of 1.006 g/mL at 25° C., and may then be encapsulated into soft elastic gelatin capsules (Capsugel, Inc.) to afford a dose of 30 mg/mL (Table 1).

TABLE 1

| Ingredient | w-% |
|---|---|
| T-62 | 6.08 |
| propylene glycol monocaprylate (Capryol 90 ®) | 43.92 |
| caprylocaproyl macrogol-8 glycerides (Labrasol ®) | 16.70 |
| super refined soybean oil (USP) | 25.00 |
| polysorbate 80 (Crillet 4 HP ®) | 8.30 |
| TOTAL | 100 |

### EXAMPLE 4

### T-62 Formulation B

T-62 (dry powder) is milled using a Quadro Comil 197 with screen (2A018R01530) and impeller (2A16011730212) at 2400 rpm. The milled T-62 is then added to a mixture of super refined soybean oil (USP), propylene glycol monocaprylate (Capryol 90®), caprylocaproyl macrogol-8 glycerides (Labrasol™), and polysorbate 80 (Crillet 4 HP®) at 50-55° C. The mixture is stirred until T-62 dissolved. The mixture/solution is sparged with nitrogen throughout the process. The resulting solution may then be encapsulated into hard gelatin capsules (Capsugel) to afford a dose of 70 mg/mL (Table 2).

TABLE 2

| Ingredient | w-% |
|---|---|
| T-62 | 8.33 |
| propylene glycol monocaprylate (Capryol 90 ®) | 41.67 |
| caprylocaproyl macrogol-8 glycerides (Labrasol ®) | 16.70 |
| super refined soybean oil (USP) | 25.00 |
| polysorbate 80 (Crillet 4 HP ®) | 8.30 |
| TOTAL | 100 |

### EXAMPLE 5

### T-62 Formulation C

T-62 (dry powder) is milled using a Quadro Comil 197 with screen (2A018R01530) and impeller (2A16011730212) at 2400 rpm. The milled T-62 is then micronized using a Glen Mills Jet Mill with nitrogen as the propellant. T-62 is passed through the Jet Mill twice to reduce the particle size to a mean diameter of 12.2 μm. The micronized T-62 is added to a mixture of super refined soybean oil (USP) with propylene glycol monocaprylate (Capryol 90®) caprylocaproyl macrogol-8 glycerides (Labrasol®), and polysorbate 80 (Crillet 4 HP®) at 50-55° C. using a propeller type mixer to dissolve T-62. The mixture/solution is sparged with nitrogen throughout the process. The resulting solution may then be encapsulated into hard gelatin capsules (size 00 Capsules, obtained from Capsugel®) to afford a dose of 70 mg/mL (Table 3).

TABLE 3

| Ingredient | w-% |
|---|---|
| T-62 | 8.33 |
| propylene glycol monocaprylate (Capryol 90 ®) | 41.67 |
| caprylocaproyl macrogol-8 glycerides (Labrasol ®) | 16.70 |
| super refined soybean oil (USP) | 25.00 |
| polysorbate 80 (Crillet 4 HP ®) | 8.30 |
| TOTAL | 100 |

### EXAMPLE 6

### T-62 Formulation D

T-62 (dry powder) is milled using a Quadro Comil 197 with screen (2A018R01530) and impeller (2A16011730212) at 2400 rpm. The milled T-62 is then added to a mixture of propylene glycol monocaprylate (Capryol 90®), caprylocaproyl macrogol-8 glycerides (Labrasol®), and polysorbate 80 (Crillet 4 HP®) at 45±5° C. using a propeller type mixer to dissolve T-62. The mixture/solution is sparged with nitrogen throughout the process. Super refined soybean oil (USP) is then added with continued mixing. The solution is allowed to return to room temperature, and then pumped though a 5 μm Meissner filter capsule. The resulting solution may then be encapsulated into soft elastic gelatin capsules (Capsugel, Inc.) to afford a dose of 100 mg/mL (Table 4).

TABLE 4

| Ingredient | w-% |
|---|---|
| T-62 | 8.33 |
| propylene glycol monocaprylate (Capryol 90 ®) | 41.67 |
| caprylocaproyl macrogol-8 glycerides (Labrasol ®) | 29.20 |
| super refined soybean oil (USP) | 12.50 |
| polysorbate 80 (Crillet 4 HP ®) | 8.30 |
| TOTAL | 100 |

### EXAMPLE 7

### T-62 Formulation E

T-62 (dry powder) is milled using a Quadro Comil 197 with screen (2A018R01530) and impeller (2A16011730212) at 2400 rpm. The milled T-62 is then added to a mixture of super refined soybean oil (USP) with propylene glycol monocaprylate (Capryol 90®), caprylocaproyl macrogol-8 glycerides (Labrasol®), polysorbate 80 (Crillet 4 HP®) and ethanol at 45±5° C. using a propeller type mixer to dissolve T-62. The mixture/solution is sparged with nitrogen throughout the process. Super refined soybean oil is then added with continued mixing. The solution is allowed to return to room temperature, and then pumped though a 5 μm Meissner filter capsule. The resulting solution may then be encapsulated into soft elastic gelatin capsules (size 00 Capsules, obtained from Capsugel®) to afford a dose of 100 mg/mL (Table 5).

TABLE 5

| Ingredient | w-% |
|---|---|
| T-62 | 8.33 |
| propylene glycol monocaprylate (Capryol 90 ®) | 41.67 |
| caprylocaproyl macrogol-8 glycerides (Labrasol ®) | 21.20 |
| super refined soybean oil (USP) | 12.50 |
| polysorbate 80 (Crillet 4 HP ®) | 8.30 |
| ethanol | 8.00 |
| TOTAL | 100 |

What is claimed is:

1. A method for achieving a synergistic therapeutic effect comprising alleviating pain in a patient, in need thereof, which method comprises administering to said patient synergistic amounts of an allosteric adenosine A₁ receptor enhancer, or a pharmaceutically acceptable salt thereof, and another therapeutic agent selected from the group consisting of:
(1) an opioid, or a pharmaceutically acceptable salt thereof; and
(2) an AMPA/kainate antagonist, or a pharmaceutically acceptable salt thereof.

2. A method according to claim 1, wherein the allosteric adenosine A₁ receptor enhancer is selected from the group consisting of compounds of the formulae 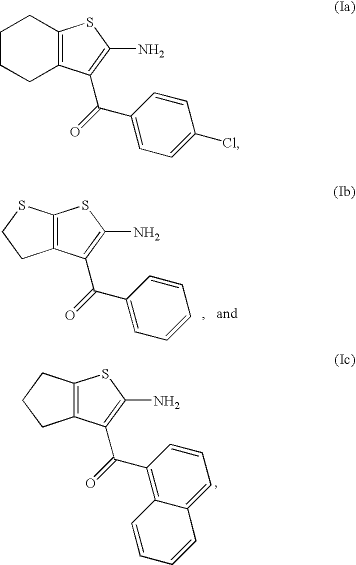
or a pharmaceutically acceptable salt thereof.

3. A method according to claim 2, wherein the other therapeutic agent is an opioid, or a pharmaceutically acceptable salt thereof.

4. A method according to claim 3, wherein the opioid is selected from the group consisting of alfentanil, allylprodine, alphaprodine, anileridine, apomorphine, apocodeine, benzylmorphine, bezitramide, buprenorphine, butorphanol, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, nalbuphine, narceine, nicomorphine, norlevorphanol, normethadone, nalorphine, normorphine, norpipanone, ohmefentanyl, opium, oxycodone, oxymorphone, papavereturn, pentazocine, phenadoxone, phenomorphan, phenazocine, phenoperidine, pholcodine, piminodine, piritramide, propheptazine, promedol, profadol, properidine, propiram, propoxyphene, remifentanyl, sufentanyl, tramadol, tilidine, naltrexone, naloxone, nalmefene, methylnaltrexone, naloxone methiodide, nalorphine, naloxonazine, nalide, nalmexone, nalbuphine, nalorphine dinicotinate, naltrindole (NTI), naltrindole isothiocyanate, (NTII), naltriben (NTB), nor-binaltorphimine (nor-BNI), β-funaltrexamine (β-FNA), cyprodime, etorphine, diprenorphine, naloxone benzoylhydrazone, bremazocine, ethylketocyclazocine, spiradoline, Met-enkephalin, Leu-enkephalin, β-endorphin, dynorphin A, dynorphin B or α-neoendorphin, or a pharmaceutically acceptable salt thereof.

5. A method according to claim 3, wherein the opioid is selected from the group consisting of hydrocodone, hydromorphone, morphine, oxycodone and oxymorphone, or a pharmaceutically acceptable salt thereof.

6. A method according to claim 3, wherein the opioid is morphine, or a pharmaceutically acceptable salt thereof.

7. A method according to claim 6, wherein the pain is postoperative pain.

8. A method according to claim 2, wherein the other therapeutic agent is an AMPA/kainate antagonist or, a pharmaceutically acceptable salt thereof.

9. A method according to claim 8, wherein the AMPA/kainate antagonist is selected from the group consisting of 2,3-dihydroxy-6-nitro-7-sulfamoylbenzo(f)quinoxaline; 6-cyano-7-nitro-quinoxaline-2,3-dione; 6,7-dinitroquinoxaline-2,3-dione; 1,4,7,8,9,10-hexahydro-9-methyl-6-nitropyrido[3,4-f]quinoxaline-2,3-dione; becampanel; talampanel; tezampanel; perampanel; and NS-1209; or a pharmaceutically acceptable salt thereof.

10. A method according to claim 8, wherein the AMPA/kainite antagonist is selected from the group consisting of 2,3-dihydroxy-6-nitro-7-sulfamoylbenzo(f)quinoxaline, becampanel, talampanel, tezampanel, perampanel and NS-1209, or a pharmaceutically acceptable salt thereof.

11. A method according to claim 8, wherein the AMPA/kainate antagonist is 2,3-dihydroxy-6-nitro-7-sulfamoylbenzo(f)quinoxaline, or a pharmaceutically acceptable salt thereof.

12. A method according to claim 11, wherein the pain is postoperative pain.

13. A method according to claim 1, wherein the allosteric adenosine A₁ receptor enhancer is a compound of the formula 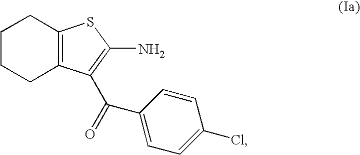
or a pharmaceutically acceptable salt thereof.

14. A method according to claim 13, wherein the other therapeutic agent is an opioid, or a pharmaceutically acceptable salt thereof.

15. A method according to claim 14, wherein the opioid is selected from the group consisting of alfentanil, allylprodine, alphaprodine, anileridine, apomorphine, apocodeine, benzylmorphine, bezitramide, buprenorphine, butorphanol, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, nalbuphine, narceine, nicomorphine, norlevorphanol, normethadone, nalorphine, normorphine, norpipanone, ohmefentanyl, opium, oxycodone, oxymorphone, papavereturn, pentazocine, phenadoxone, phenomorphan, phenazocine, phenoperidine, pholcodine, piminodine, piritramide, propheptazine, promedol, profadol, properidine, propiram, propoxyphene, remifentanyl, sufentanyl, tramadol, tilidine, naltrexone, naloxone, nalmefene, methylnaltrexone, naloxone methiodide, nalorphine, naloxonazine, nalide, nalmexone, nalbuphine, nalorphine dinicotinate, naltrindole (NTI), naltrindole isothiocyanate, (NTII), naltriben (NTB), nor-binaltorphimine (nor-BNI), β-funaltrexamine (β-FNA), cyprodime, etorphine, diprenorphine, naloxone benzoylhydrazone, bremazocine, ethylketocyclazocine, spiradoline, Met-enkephalin, Leu-enkephalin, β-endorphin, dynorphin A, dynorphin B or α-neoendorphin, or a pharmaceutically acceptable salt thereof.

16. A method according to claim 14, wherein the opioid is selected from the group consisting of hydrocodone, hydromorphone, morphine, oxycodone and oxymorphone, or a pharmaceutically acceptable salt thereof.

17. A method according to claim 14, wherein the opioid is morphine, or a pharmaceutically acceptable salt thereof.

18. A method according to claim 17, wherein the pain is postoperative pain.

19. A method according to claim 13, wherein the other therapeutic agent is an AMPA/kainate antagonist or, a pharmaceutically acceptable salt thereof.

20. A method according to claim 19, wherein the AMPA/kainate antagonist is selected from the group consisting of 2,3-dihydroxy-6-nitro-7-sulfamoylbenzo(f)quinoxaline; 6-cyano-7-nitro-quinoxaline-2,3-dione; 6,7-dinitroquinoxaline-2,3-dione; 1,4,7,8,9,10-hexahydro-9-methyl-6-nitropyrido[3,4-f]quinoxaline-2,3-dione; becampanel; talampanel; tezampanel; perampanel; and NS-1209; or a pharmaceutically acceptable salt thereof.

21. A method according to claim 19, wherein the AMPA/kainite antagonist is selected from the group consisting of 2,3-dihydroxy-6-nitro-7-sulfamoylbenzo(f)quinoxaline, becampanel, talampanel, tezampanel, perampanel and NS-1209, or a pharmaceutically acceptable salt thereof.

22. A method according to claim 19, wherein the AMPA/kainate antagonist is 2,3-dihydroxy-6-nitro-7-sulfamoylbenzo(f)quinoxaline, or a pharmaceutically acceptable salt thereof.

23. A method according to claim 22, wherein the pain is postoperative pain.

24. A method for achieving a synergistic therapeutic effect comprising alleviating acute pain, chronic pain, inflammatory pain, neuropathic pain or pain associated with migraine in a patient, in need thereof, which combination comprises synergistic amounts of an allosteric adenosine A₁ receptor enhancer of the formula 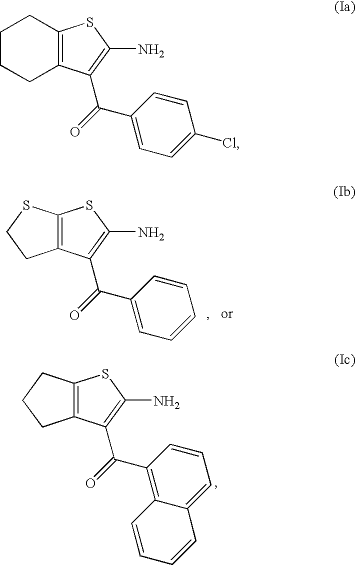
or a pharmaceutically acceptable salt thereof, and another therapeutic agent selected from the group consisting of:
(1) an opioid, or a pharmaceutically acceptable salt thereof; and
(2) an 2-amino-3-hydroxy-5-methyl-4-isoxazole-propionic acid (AMPA)/kainate antagonist, or a pharmaceutically acceptable salt thereof.

25. A method according to claim 24, wherein the opioid is selected from the group consisting of hydrocodone, hydromorphone, morphine, oxycodone and oxymorphone, or a pharmaceutically acceptable salt thereof; and the AMPA/kainite antagonist is selected from the group consisting of 2,3-dihydroxy-6-nitro-7-sulfamoylbenzo(f)quinoxaline, becampanel, talampanel, tezampanel, perampanel and NS-1209, or a pharmaceutically acceptable salt thereof.
